(19) United States
(12) Patent Application Publication
(10) Pub. No.: US 2004/0210216 A1
(43) Pub. Date: Oct. 21, 2004

(54) Spinal fixation system and method

(76) Inventors: Robert A. Farris, Cordova, TN (US); Kevin T. Foley, Germantown, TN (US); Bradley J. Coates, Rossville, TN (US); James J. Bindseil, Germantown, TN (US)

Correspondence Address: COATS & BENNETT, PLLC, P O BOX 5, RALEIGH, NC, 27602, US

(21) Appl. No.: 10/418,030

(22) Filed: Apr. 17, 2003

(51) Int. Cl. A61B 17/70; A61B 17/56

(52) U.S. Cl. 606/61; 606/72; 606/53; 623/17.11; 623/908

(57) ABSTRACT

A device and method of attaching an elongated rod to different vertebral members to form a spinal fixation system. The device comprises a securement member having a first section that is positioned within the vertebral member. A second section extends laterally outward from the first section. A receiving member is mounted to the second section to receive the elongated rod. Receiving member includes a receiver for receiving and attaching to the second section. The receiving member is laterally movable along the lateral section for positioning the elongated rod relative to the spine of the patient. The length of the second section provides for mounting the first section at a variety of anatomically conducive locations on the vertebral member while supporting the elongated rod.

### BACKGROUND

One technique for spinal fixation includes immobilizing the spine by a spine rod that extends along the back generally parallel to the spine. The rod is attached to the vertebral members with bone screws. In one embodiment, the rod is directly connected to the bone screw. In another embodiment, a receiver is connected to the bone screw for receiving and positioning the rod relative to the spine.

It is important that the rod is accurately positioned to extend along the spine. The rod is secured along the spine by a number of different bone screws mounted along the rod length to different vertebral members. In one embodiment, a bone screw is placed in each vertebral member and connected to the rod. A drawback of previous mounting systems is that the screws must be accurately placed in each of the different vertebral members to accommodate the rod. The rod cannot be connected if the screws are not properly aligned along the spine.

Previous systems do not provide adjustability in positioning the rod relative to the bone screw. Therefore, the exact placement of the screw is important to properly align the rod along the spine. Bone screw placement is often made difficult because of the anatomic structure of the vertebral members. The various undulations on the surface of the vertebral members may require either excessive amounts of skill by the surgeon for proper placement, additional bone removal from the vertebral members to properly place the bone screws, or an insecure fastening of the bone screw of the vertebral member.

### SUMMARY

The present invention is directed to devices and methods for positioning a rod along the spine. The devices and methods permit angulation between a fastener or securement member and the rod, as well as independently permitting medial/lateral offset between the two. In one embodiment, the device includes a securement member having a first section for placement into a vertebral member and a second section extending from the first section. A receiving member is mountable on the second section and has a channel for receiving the elongated rod. A securing means secures the elongated rod within the receiving member. The device is constructed such that the first section can be placed in the vertebral member at a variety of positions relative to a centerline of the spine and allow for positioning of the rod. The differences in placement of the securement members is compensated for by moving the receiving member along the second section.

The present invention further includes a method of connecting an elongated rod along the spine. One embodiment of using the device includes attaching the first securement member to a first vertebral member with a first section positioned within the first vertebral member at a first distance from a centerline of the spine and a first lateral section extending above the first vertebral member. A second securement member is attached to a second vertebral member at a second distance from the centerline of the spine with a first section positioned within the first vertebral member and a second lateral section extending above the first vertebral member. The first securement member is positioned a different distance from the centerline then the second securement member. The elongated rod is positioned parallel to the centerline of the spine with the elongated rod crossing the first lateral section a point from the first section and crossing the second lateral section.

### BRIEF DESCRIPTION OF THE DRAWINGS

FIG. 1 is a perspective view of one embodiment of the present invention;

FIG. 2 is an exploded side view of one embodiment of the present invention;

FIG. 3 is a side view of one embodiment of the present invention;

FIG. 4 is a side view of another embodiment of the present invention;

FIG. 5 is a plan view of one embodiment of a spinal fixation system;

FIG. 6 is side view of another embodiment of the present invention including a partial cross-sectional view of a locking mechanism; and

FIG. 7 is a side view of another embodiment similar to FIG. 6.

### DETAILED DESCRIPTION

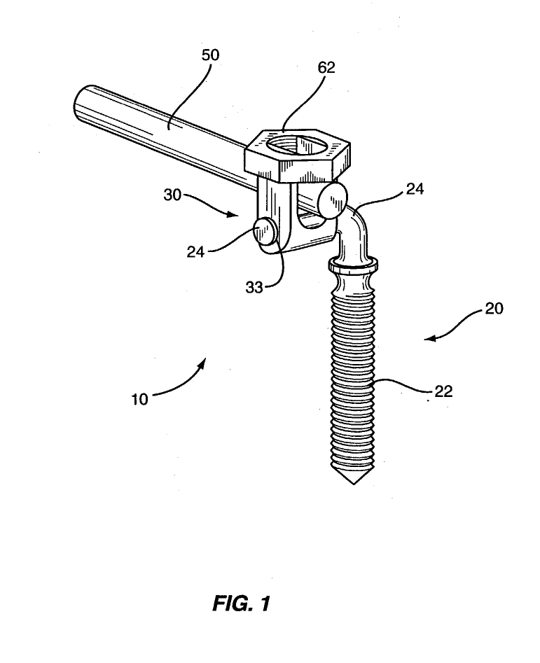

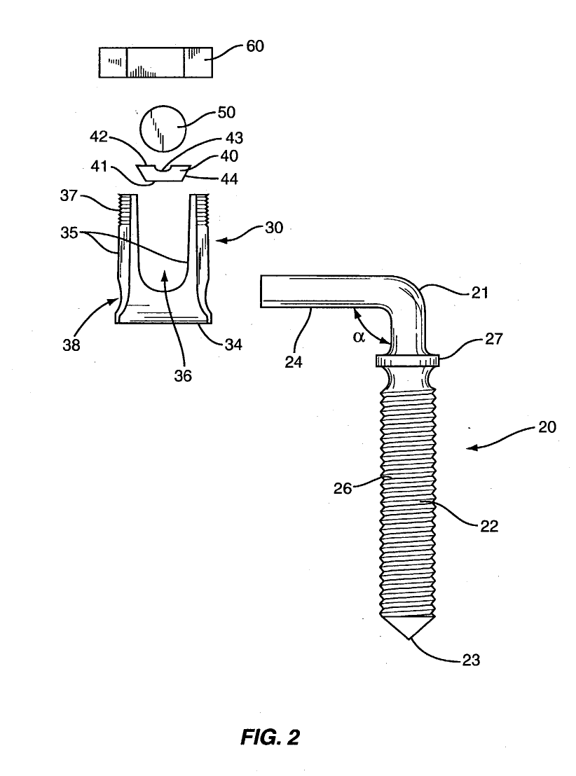

The present invention is directed to devices and methods of attaching an elongated rod to different vertebral members to form a spinal fixation system. The device, generally illustrated as 10 in FIG. 1, comprises a securement member 20 having a first section 22 positioned within the vertebral member. A second section 24 extends outward and at an angle from the first section 22. A receiving member 30 is mounted to the second section 24 to receive the elongated rigid member or rod 50. Receiving member 30 includes an attachment section 38 (FIG. 2) for receiving and attaching to the second section 24. The receiving member 30 is laterally movable along the second section 24 for positioning the elongated rod 50 relative to the spine of the patient. The length of the second section 24 provides for mounting the first section 22 at a variety of anatomically conducive locations on the vertebral member and supporting the elongated rod 50. The device 10 permits angulation between the securement member 20 and a rod 50, as well as independently permitting medial/lateral offset between the two.

The securement member 20 anchors the elongated rod 50 to the vertebral members. In one embodiment, securement member 20 includes a first section 22 that mounts to the vertebral member, and a second section 24 that mates with the receiving member 30. First section 22 may have a variety of orientations to attach to the vertebral member. In one embodiment, threads 26 (FIG. 2) are positioned about at least a portion of the first section 22. The first section end may include a pointed tip 23 to facilitate insertion and mounting within the vertebral member. In one embodiment, a flange 27 (FIG. 2) extends radially outward from the first section 22. The flange 27 may be positioned to control the depth of insertion of the first section 22 into the vertebral member.

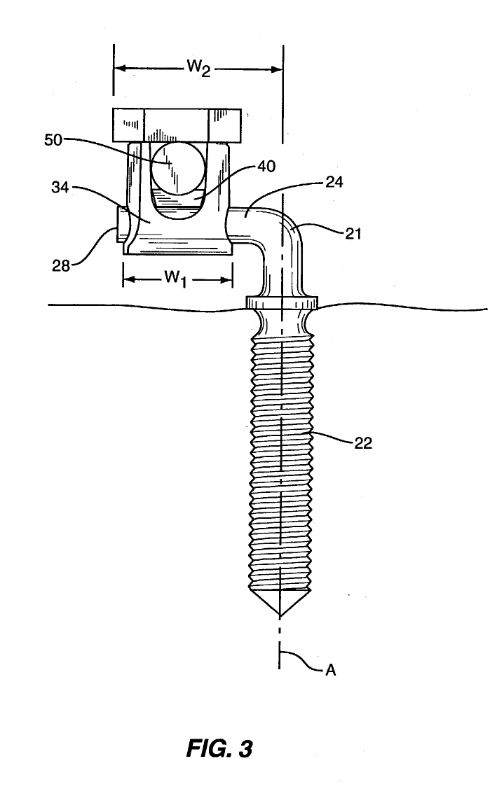

Second section 24 extends outward from first section 22 at a predetermined angle and over the vertebral member 100 as illustrated in FIG. 3. In one embodiment, second section 24 extends outward from the first member at about 90 degrees, as illustrated by angle α in FIG. 2. Angle α may be in the range of about 10° to about 170° or about 30° to about 150°, or about 70° to about 110°. In one embodiment, angle α is about 80°.

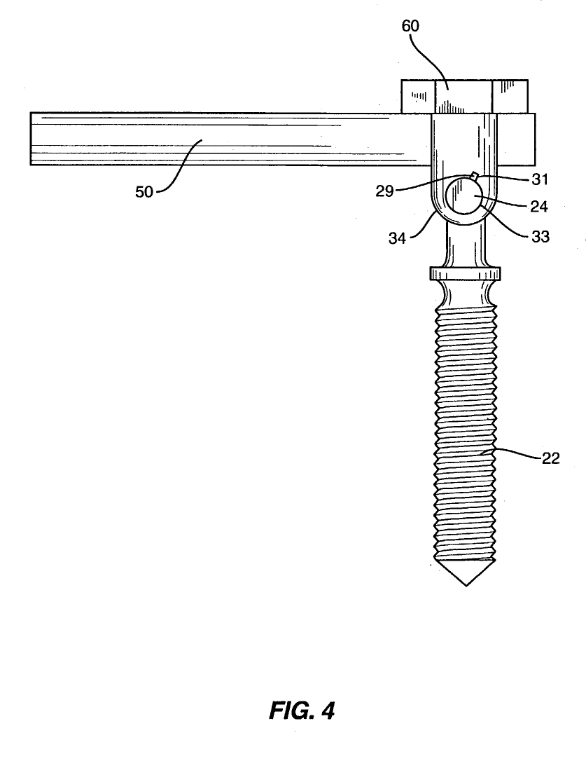

Second section 24 may have a variety of cross-sectional shapes. In one embodiment as best illustrated in FIG. 1, the cross-sectional shape is substantially circular. In another embodiment as illustrated in FIG. 4, the second section 24 and the attachment section 38 are keyed. In one embodiment, key 29 extends outward from the second section 24 for mating with the receiving member 30. The key 29 may be positioned at various locations about the second section 24, and may have a variety of shapes and sizes. Key 29 is sized to mate with and prevent the receiving member 30 from rotating about the axis of the second section 24. In one embodiment, two or more keys 29 extend outward from the second section 24. The one or more keys 29 may be positioned at a variety of positions about the second section 24. In one embodiment, key 29 extends along the length of the second section 24. In one embodiment, key 29 extends a limited distance from the distal end of the second section 24. In another embodiment, second section 24 includes an indent (not illustrated) that extends into the cross-sectional shape (i.e., opposite the key 29). A key 29 is positioned on the receiving member 30 that aligns with the indent In one embodiment, second section may include two or more indents located at variations positions. In one embodiment, the keys 29 and indents are substantially linear. In another embodiment, the keys 29 and indents are helical. In one embodiment, attachment section 38 includes one or more indents 31 for receiving the one or more keys 29 of the second section 24. In another embodiment, attachment section 38 includes one or more keys that mount within one or more indents of the second section 24.

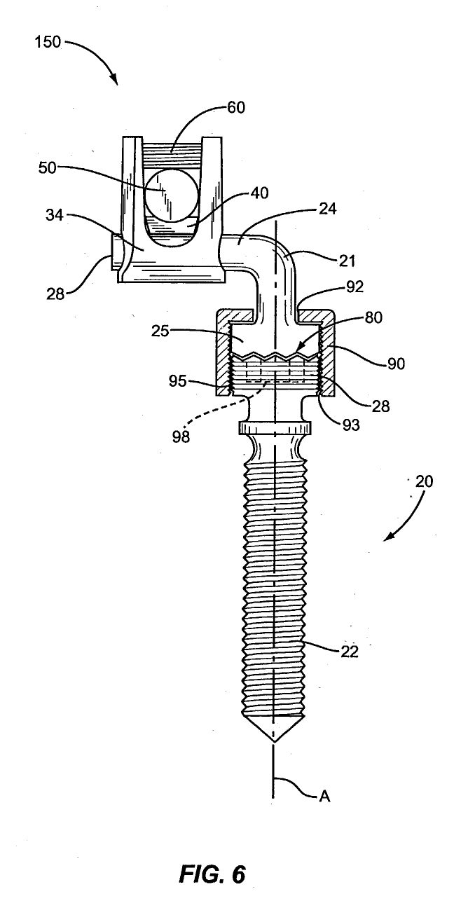

In one embodiment, the first section 22 and second section 24 are constructed of a single member. Referring to FIG. 2, the single member includes a bent section 21 that extends into the first and second sections 22, 24 respectively. In another embodiment, the first section and second sections 22, 24 are constructed of different members. In one embodiment, first section 22 and second section 24 are separate as illustrated in FIG. 6.

Receiving member 30 connects the elongated rod 50 to the securement member 20. In one embodiment referring to FIG. 2, receiving member 30 includes a receiver attachable with the securement member 20, and a channel 36 for receiving the elongated rod 50. In one embodiment, attachment section 38 includes a base 34 that extends between sidewalls 35. Base 34 includes an opening 33 (FIG. 1) through which the securement member second section 24 extends. In one embodiment as illustrated in FIG. 4, opening 33 is positioned completely within the base 34 and surrounds the second section 24. In this embodiment, the second section 24 does not directly contact the fastener 40 and/or elongated rod 50. In another embodiment as illustrated in FIG. 3, the second section 24 extends beyond the base 34 to directly contact the fastener 40 and/or elongated rod 50.

In one embodiment as illustrated in FIG. 1, the opening 33 and cross-sectional shape of the second section 24 have corresponding shapes such that the receiving member 30 can rotate about the second section 24. In one embodiment, the opening 33 and cross-sectional shape of the second section 24 are both circular in shape. In one embodiment, the receiving member rotates completely around the second section (i.e., 360 degrees). In one embodiment, the opening and second section 24 are shaped to provide a limited amount of rotation. In one embodiment, the receiving member 30 can rotate about the second section 24 about 180 degrees.

In one embodiment as illustrated in FIG. 3, the base 34 has a width w1 less than the width w2 of the second section 24. The difference in widths allows for the receiving member 30 to be positioned at different locations along the second section 24. Therefore, the elongated rod 50 can be positioned at different distances from a centerline A of the first section 22 to allow for variable relative positions between the securement member 20 and elongated rod 50.

Referring to FIG. 2, the channel 36 receives the elongated rod 50. In one embodiment, channel 36 is formed between opposing sidewalls 35. The channel 36 has open ends through which the elongated rod 50 extends. In one embodiment, sidewalls 35 have a length greater than the diameter of the elongated rod 50. Outer edges of the sidewalls 35 may include threads 37 for receiving the fastener 60. Alternately, sidewalls 35 may include inner threads that cooperate with external threads of an internal screw member that would replace an outer nut or locking member 60 as illustrated in FIG. 6.

In one embodiment, a spacer 40 is positioned within the channel 36. In one embodiment, spacer 40 is positioned between the elongated rod 40 and the second section 24. First side 41 and second side 42 of spacer 40 may be shaped to increase contact with the second section 24 of member 20 and rod 50, respectively. The shaped surfaces of sides 41, 42 increase friction between the adjacent components when the locking member 60 is tightened onto the receiving member 30, thereby decreasing the likelihood of relative motion between the components and second section 24 of securement member 20. In one embodiment, spacer 40 includes a first side 41 having a shape corresponding to the top of the second section 24. A second side 42 has an indent 43 shaped to conform to the outer surface of the elongated rod 50. Outer side 44 may be sized to fit within channel 36. In another embodiment, first side 41 contacts the lower portion of the channel 36 and does not contact the second section 24. In one embodiment, first side 41 is shaped to conform to the lower portion of the channel 36.

In one embodiment, a locking member 60 is affixed to the receiving member 30 to maintain the elongated rod 50 within the channel 36. In one embodiment, locking member 60 has threads 62 on an inner edge that mate with threads 37 on the sidewalls 35 as illustrated in FIG. 1. In another embodiment, locking member 60 includes exterior threads that mate with threads on the interior edge of the sidewalls 35 as illustrated in FIG. 6. In one embodiment, the locking mechanism 60 directly contacts the elongated rod 50 as illustrated in FIG. 3. In one embodiment, the locking mechanism 60 extends beyond the top edges of the sidewalls 35 when the locking mechanism 60 is secured to the receiving member 30.

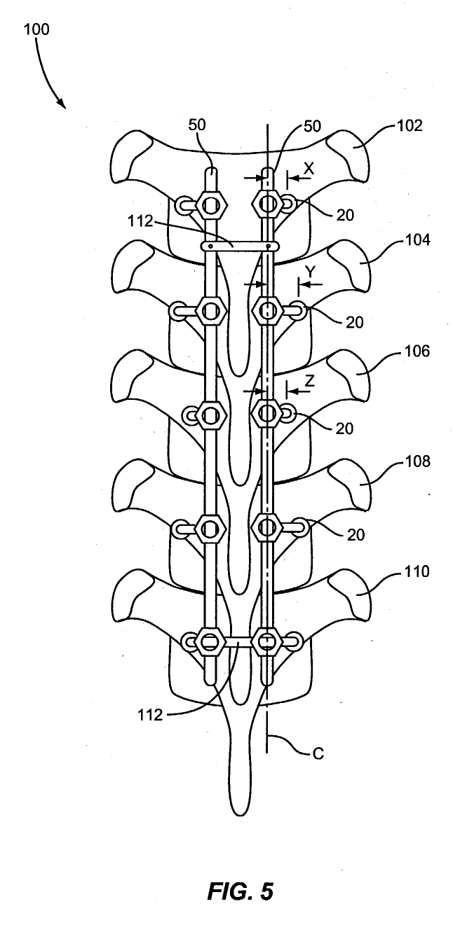

FIG. 5 illustrates one embodiment of a spinal fixation system 100 having one or more vertebral rods 50 extending along vertebral members 102, 104, 106, 108, 110. The first section 22 of the securement members 20 may be positioned at different locations from the centerline C of the elongated rod 50. Different relative positioning of the first sections 22 may be necessary because of the structure of the individual vertebral members facilitate a secure attachment. In the embodiment illustrated in FIG. 5, for example, a center of first section 22 is placed a distance x from centerline C in vertebral member 102, a center of first section 22 is placed a distance y from centerline C in vertebral member 104, and a center of first section 22 is placed a distance z from centerline C in vertebral member 106. Each distance x, y, z may be the same or different distances. The receiving member 30 is movable along the second sections 24 such that the rod 50 can be maintained at a predetermined position despite the position of the first sections 22. In one embodiment, the different securement members 20 are mounted at different angles within the vertebral members. Additionally, spinal fixation system 100 may include one or more lateral connectors 112 for relatively securing adjacent rods 50. The invention permits angulation between the different securement members 20 while providing for lateral and medial offset.

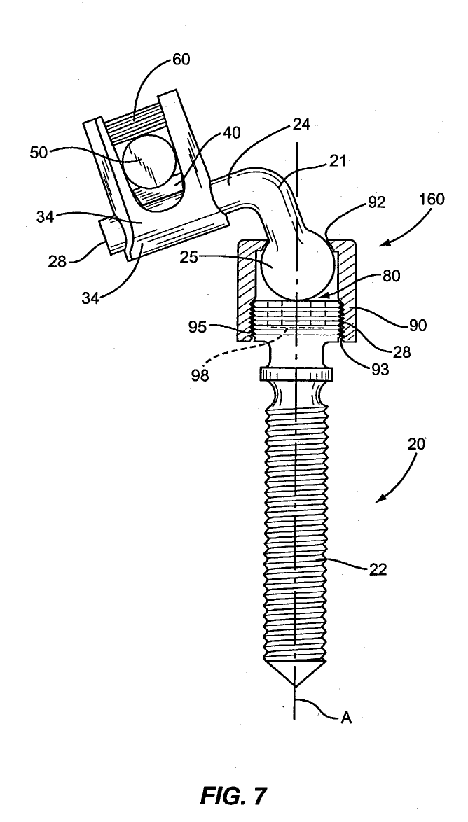

In another embodiment illustrated in FIGS. 6 and 7, fixation systems 150, 160 include securement member 20 having an independent bone attachment or first section 22 and rod attachment or second section 24 joined at mating interface 80. Mating interface 80 may include any interface that joins first section 22 and second section 24. For instance, mating interface 80 may include flat surfaces, a flat or partially round surface with a round surface, or corresponding undulating surfaces that allow second section 24 to be positioned in predetermined relative rotational positions with respect to first section 22. In one embodiment, mating sections 25, 28 are positioned at mating ends of the first and second sections 22, 24, respectively to form the mating interface 80. The mating sections 25, 28 may have a larger cross-sectional area than the remainder of the first and second sections 22, 24.

Locking mechanism 90, such as a collet, fixes the second section 24 to the first section 22 to prevent relative movement between the sections. Locking mechanism 90 may also include a sleeve, a clamp, a crimp, a detent, glue, a weld, or any other mechanical or chemical mechanism for affixing the first section 22 and the second section 24. Locking mechanism may further have a variety of different exterior shapes, including square, circular, and polygonal. In one embodiment, an aperture 92 is sized to extend around the second section 24 such that second section 24 is clamped to first section 22 as locking mechanism 90, having internal threads 93, is screwed on to mating section 28, having corresponding external threads 95. In one embodiment, aperture 92 has a smaller cross-sectional size than first mating portion 25 to maintain the locking mechanism 90 connected with the second section 24.

In the embodiment of FIG. 6, receiving member 30 is positioned onto the second section 24, and fixed relative to the second section 24 and rod 50 by locking member 60, such as, an internal set screw.

In the embodiment of FIG. 6, the separable configuration of first section 22 and second section 24 allows for first section 22 to be inserted without requiring clearance for rotation of the laterally extending second section 24. For example, in this embodiment, first section 22 may include a driver interface 98 shaped to correspond to a driver for inserting securement member 20. Driver interface 98 may be an internal or an external feature, such as a hexagonal shape, a cross shape, a rectangular shape, a triangular shape, or any other shape suitable for driving the first section 22 into bone.

The term “vertebral member” is used generally to describe the vertebral geometry comprising the vertebral body, pedicles, lamina, and processes. The securement member 20 may be sized and shaped, and have adequate strength requirements to be used within the different regions of the vertebra including the cervical, thoracic, and lumbar regions.

The present invention may be carried out in other specific ways than those herein set forth without departing from the scope and essential characteristics of the invention. In one embodiment, the receiving member has a saddle shape. The present embodiments are, therefore, to be considered in all respects as illustrative and not restrictive, and all changes coming within the meaning and equivalency range of the appended claims are intended to be embraced therein.

What is claimed is:

1. A spinal fixation system comprising:
a securement member having a first section for placement into a vertebral member and a second section extending from the first section at a predetermined angle;
a receiving member having a receiving opening and opposing sidewalls forming a channel having an open side, the receiving opening sized to receive the second section and movable along the second section, and at a variety of angular orientations relative to the second section;
an elongated rod sized to fit within the channel; and
a securing means for securing the elongated rod within the receiving member.

2. The system of claim 1, wherein the predetermined angle is in the range of about 10° to about 170°.

3. The system of claim 1, wherein the second section has a cross-sectional shape corresponding to a shape of the receiving opening.

4. The system of claim 1, wherein the securement member is constructed of a single member having an angled configuration defining the first section and the second section.

5. The system of claim 4, wherein the first section comprises threads for engaging into the vertebral members.

6. The system of claim 1, wherein the predetermined angle is in the range of about 70° to about 110°.

7. The system of claim 1, wherein the sidewalls have a length greater than a cross-sectional dimension of the elongated rod.

8. The system of claim 1, further comprising a spacer positioned between the second section and the elongated rod, the spacer having a first side shaped to correspond with an outer surface of the second section and a second side shaped to correspond to an outer surface of the elongated rod.

9. The system of claim 1, wherein a width of the receiving member is less than a distance between a distal end of the second section and a proximal end of the second section.

10. The system of claim 1, wherein the first section is a separate element and attached to the second section.

11. The system of claim 1, wherein the predetermined angle is about 80°.

12. The system of claim 1, wherein the receiving opening and the second section are keyed.

13. A device for securing an elongated rod to a vertebral member comprising:
a securement member having a first section, a bend section, and a second section, the second section having a distal end; and
a receiving member sized to mate with the second section and movable along the second section between the distal end and the bend section and rotatable about the second section, the receiving member further comprising a channel to receive the elongated rod.

14. The device of claim 13, wherein the bend section connects the first section relative to the second section at an angle in the range of about 70° to about 110°.

15. The device of claim 13, wherein a width of the receiving member is less than a distance between the distal end of the second section and the bend section.

16. The device of claim 13, further comprising a locking member affixable to the receiving member to maintain the elongated rod in a predetermined position relative to the securement member.

17. The device of claim 13, wherein the second section and the receiving member are each keyed.

18. A spinal fixation system comprising:
a first section having a substantially linear orientation with exterior threads extending along a portion to mount within a vertebral member, the first section having a first mating portion;
a second section having a second mating portion;
a locking mechanism adjustable between a first orientation in which the first section is separate from the second section and a second orientation to extend around the first mating portion and the second mating portion and position the first mating portion in contact with the second mating portion with the second section extending from the first section at a predetermined angle;
a receiving member having a receiving opening and opposing sidewalls forming a channel, the receiving opening sized to receive the second section and movable along the second section; and
an elongated rod sized to fit within the channel.

19. The system of claim 18, wherein the predetermined angle is in the range of about 10° to about 170°.

20. The system of claim 18, wherein the predetermined angle is in the range of about 70° to about 110°.

21. The system of claim 18, wherein the predetermined angle is about 80°.

22. The system of claim 18, wherein the first section and the second section each include external threads that mate with internal threads of the locking mechanism.

23. The system of claim 18, wherein the first mating portion and the second mating portion each include complimentary undulating surfaces that mate together in the second orientation to align the second section relative to the first section.

24. The system of claim 18, wherein the first section includes a proximal end having a driver interface and a distal end that mounts within a vertebral member.

25. The system of claim 18, wherein the second section and the receiving member are each keyed.

26. The system of claim 18, wherein the locking mechanism includes an aperture through which the second section extends to attach the locking mechanism to the second section in the first orientation.

27. A spinal fixation system comprising:
a first and a second elongated rod;
a first and second plurality of securement members each having a first section that is positioned within a vertebral member, and a second section affixed to the first section at a predetermined relative angle between about 30° and about 150°;
a plurality of receiving members each mountable to respective ones of the second sections and each having a channel formed by a pair of sidewalls and a base extending between the sidewalls, the base having a width less than the second section, and the receiving member having an open end through which the elongated rod is extendable, each receiving member further comprising a receiving opening within the base to receive the respective second sections;
a plurality of spacers each sized, to fit within respective ones of the channels and each having a first side that is seatable against the respective second section and a second side that is seatable against the respective elongated rod; and
a plurality of locking members each affixable to respective ones of the plurality of receiving members to secure the elongated rods within the channels.

28. The system of claim 27, wherein the predetermined relative angle is between about 70° and about 110°.

29. The system of claim 27, wherein the predetermined relative angle is about 80°.

30. The system of claim 27, wherein at least one of the plurality of second sections and at least one of the plurality of receiving members are keyed.

31. The system of claim 27, further comprising a lateral connector affixable between the first elongated rod and the second elongated rod.

32. A spinal fixation system, comprising:
a first fastener portion affixable to a bone and having a first mating section;
a second fastener portion having a second mating section affixable with the first mating section, wherein the second fastener portion is extendable from the first fastener portion at a predetermined angle relative to a longitudinal axis of the first fastener portion;
a first elongated rigid member; and
a receiver member having a first receiver portion connectable with the second fastener portion and a second receiver portion connectable with the first elongated rigid member.

33. The system of claim 32, further comprising the second mating section affixable with the first mating section in one of a plurality of predetermined positions.

34. The system of claim 32, further comprising the first mating section having a first mating interface and the second mating section having a second mating interface corresponding to the first mating interface such as to substantially fix the second mating section in one of a plurality of predetermined positions relative to the first mating section when the second mating section is affixed to the first mating section.

35. The system of claim 32, further comprising a first locking mechanism adjustably positionable with respect to the receiver member, wherein the first locking mechanism has a first position that allows relative movement between the first elongated rigid member or the second fastener portion and the receiver member and a second position that fixedly secures at least one of the first elongated rigid member or the second fastener portion and the receiver member.

36. The system of claim 32, further comprising a second locking mechanism positionable adjacent to the first mating section and the second mating section, wherein the second locking mechanism has a first position that allows relative movement between the first mating section and the second mating section and a second position that fixedly secures the first mating section and the second mating section.

37. The system of claim 32, wherein the receiver member is rotatable and translatable relative to the second fastener portion.

38. The system of claim 32, wherein the predetermined angle is between about 30 degrees and about 150 degrees.

39. The system of claim 32, further comprising a second elongated rigid member and a lateral connector affixable between the first elongated rigid member and the second elongated rigid member.

40. The system of claim 32, wherein the first mating section has a surface selected from the group consisting of a substantially flat surface, a substantially rounded surface, and an undulating surface.

41. A method of attaching an elongated rod along a spine comprising the steps of:
attaching a first securement member to a first vertebral member by inserting a first mounting section to the first vertebral member and positioning a first extended section above the first vertebral member;
attaching a second securement member to a second vertebral member by inserting a second mounting section to the second vertebral member and positioning a second extended section above the second vertebral member;
laterally adjusting a first receiving member along the first extended section a first distance from a centerline of the spine;
laterally adjusting a second receiving member along the second extended section a second distance from the centerline of the spine with the second distance being different that the first distance; and
positioning the elongated rod relative to the spine and within the first receiving member and the second receiving member.

42. The method of claim 41, further comprising the step of rotating the first receiving member about the first extended section and maintaining the first receiving member at the first distance from the centerline of the spine.

43. The method of claim 41, further comprising attaching the first mounting section to the first vertebral member at a first angle relative to the centerline of the spine, and attaching the second mounting section to the second vertebral member at a second angle relative to the centerline of the spine with the first angle being different than the second angle.
